(19) United States
(12) Patent Application Publication
(10) Pub. No.: US 2023/0277439 A1
(43) Pub. Date: Sep. 7, 2023

(54) COMPOSITIONS AND METHODS OF USE FOR THE TREATMENT OF SKIN

(71) Applicants: CryoGen, LLC, Raleigh, NC (US); Centro de Biotecnología Santer SC, Zapopan (MX)

(72) Inventors: David Uribe Rodriguez, Zapopan (MX); Jose Alejandro Robert Montenegro, Zapopan (MX); Dora Yuridia Gutierrez Rico, Zapopan (MX); Miguel Ernesto Salcedo Barba, Zapopan (MX); Reyna Lucia Barajas Torres, Zapopan (MX); Valeria Margarita Criollo Vinueza, Raleigh, NC (US); Ronnie Al Thompson, Raleigh, NC (US); lliany Annel Martinez Alvarez, Guadalajara (MX)

(21) Appl. No.: 18/116,758

(22) Filed: Mar. 2, 2023

Related U.S. Application Data
(60) Provisional application No. 63/315,600, filed on Mar. 2, 2022.

(51) Int. Cl. A61K 8/98; A61K 35/28; A61Q 19/00; A61P 17/02; A61K 45/06; A61Q 17/04

(52) U.S. Cl. CPC A61K 8/981; A61K 35/28; A61K 45/06; A61P 17/02; A61Q 17/04; A61Q 19/00; A61K 9/0014

(57) ABSTRACT

Compositions for the cosmetic and/or therapeutic treatment of skin are provided. Methods for preparing the compositions and methods for using the compositions for the treatment of skin are also provided.

### PRIOR RELATED APPLICATION

This application claims the benefit of and priority to U.S. Provisional Application No. 63/315,600, filed Mar. 2, 2022. This provisional application is incorporated by reference in its entirety.

### FIELD

The present disclosure relates to compositions for the treatment of skin. The disclosure also generally relates to methods of using the disclosed compositions for the dermatologic or cosmetic treatment of skin.

### BACKGROUND OF THE INVENTION

Skin is the largest organ of the human body and functions as a barrier to microbial infection and to the elements. Proper skin care, therefore, is important for both health and cosmetic reasons. An injury or other damage to the skin can affect the skin’s appearance and its ability to function as a barrier, exposing the body to microbial infections.

Various skin conditions, such as rashes, dermatitis, eczema, psoriasis, acne, cellulitis, rosacea may occur throughout an individual’s life. In addition, skin changes as an individual ages. The effect of aging on the skin may include an increase in wrinkles, dryness, and age spots; a thinning of the skin, resulting in less plump or smooth skin; and an increase in healing time following injury. Any of these skin conditions may affect not only the individual’s self-confidence, but also how other people perceive the individual.

Certain compositions and methods are currently available for skin treatment. However, there remains a need for improved compositions and methods for treating skin and maintaining or improving its health and appearance. The present disclosure provides such improved compositions and methods.

### BRIEF SUMMARY OF THE INVENTION

In various aspects, the inventions disclosed herein may include, but need not be limited to, any one or more of the following embodiments:

In one aspect, the disclosure provides a composition for cosmetic or therapeutic use that includes exosomes, one or more growth factors, optionally DMDM hidantoin, optionally phenoxyethanol, optionally benzyl alcohol, and fragments of mesenchymal stem cells (MSCs). In some embodiments, the composition comprises MSC conditioned media, including exosomes, one or more growth factors, and fragments of MSCs. In some embodiments, the composition includes exosomes, one or more growth factors, DMDM hidantoin, phenoxyethanol, benzyl alcohol, and fragments of MSCs. In certain embodiments of the compositions, these ingredients are present in the composition at about 0.1% w/w to about 40% w/w of the composition. In some embodiments, the composition comprises DMDM hidantoin at 0.2%, phenoxyethanol at 0.3%, and benzyl alcohol at 0.3% w/w of the composition. In some embodiments, the exosomes are present at a concentration of about 55 exosomes/µL. In some embodiments, the growth factors comprise VEGF, FGF, IL-6, CTGF, PDGF, PGE2, IL-10, HLA-G5, galectin 1, LIF, MHCII, EGF, TGF-β, IGF-1, KGF, SDF-1, MIP-1a, MIP-1b, SDF-1, or HGF, or a combination thereof. In certain embodiments, the compositions are stable and overcome the need for a cold chain network and lyophilization.

In another aspect, the disclosure provides a composition for cosmetic use, wherein the composition comprises double distilled water, one or more growth factors, placenta hydrolyzate, portulaca oleracea DNA, a water soluble emollient, cellulose, phenoxyethanol, and a fragrance. In certain embodiments of the compositions, these ingredients are present in the composition at about 0.5% w/w to about 20% w/w of the composition. In some embodiments, the composition comprises the one or more growth factors at 20% w/w of the composition, placenta hydrolyzate at about 5% w/w of the composition, portulaca oleracea DNA at about 3% w/w of the composition, and phenoxyethanol at about 0.5% w/w of the composition. In certain embodiments, the composition has a pH of about 6.

In another aspect, the disclosure provides a composition for cosmetic use, wherein the composition comprises double distilled water, one or more growth factors, portulaca oleracea extract, a water soluble emollient, an exopolysaccharide, collagen, Sodium L-Pyrrolidonecarboxylate (Sodium PCA or PcaNa), cellulose, elastin, thriethanolamine, phenoxyethanol, and a fragrance. In certain embodiments of the compositions, these ingredients are present in the composition at about 0.5% w/w to about 20% w/w of the composition. In some embodiments, the composition comprises the one or more growth factors at about 20% w/w of the composition, portulaca oleracea extract at about 10% w/w of the composition, an exopolysaccharide at about 3% w/w of the composition, collagen at about 1% w/w of the composition, elastin at about 1% w/w of the composition, and phenoxyethanol at about 0.5% w/w of the composition. In certain embodiments, the composition has a pH of about 6.5.

In another aspect, the disclosure provides a composition for cosmetic use, wherein the composition comprises double distilled water, one or more growth factors, portulaca oleracea extract, vegetable glycerin, polysorbate 20, portulaca oleracea DNA, phenoxyethanol, and a fragrance. In certain embodiments of the compositions, these ingredients are present in the composition at about 0.5% w/w to about 10% w/w of the composition. In some embodiments, the composition comprises the one or more growth factors at about 10% w/w of the composition, portulaca oleracea extract at about 10% w/w of the composition, portulaca oleracea DNA at about 3% w/w of the composition, and phenoxyethanol at about 0.5% w/w of the composition. In certain embodiments, the composition has a pH of about 6.5.

In another aspect, the disclosure provides a composition for cosmetic use, wherein the composition comprises double distilled water, one or more growth factors, portulaca oleracea extract, a water soluble emollient, portulaca oleracea DNA, collagen, sodium PCA, cellulose, elastin, phenoxyethanol, and a fragrance. In certain embodiments of the compositions, these ingredients are present in the composition at about 0.5% w/w to about 10% w/w of the composition. In some embodiments, the composition comprises the one or more growth factors at about 10% w/w of the composition, collagen at about 3% w/w of the composition, sodium PCA at about 3% w/w of the composition, elastin at about 2% w/w of the composition, and phenoxyethanol at about 0.5% w/w of the composition. In certain embodiments, the composition has a pH of about 6.5.

In another aspect, the disclosure provides a composition for cosmetic use, wherein the composition comprises double distilled water, one or more growth factors, lauryl glucoside, disodium cocoamphodiacetate, ethoxylated lanonin, cellulose, phenoxyethanol, and a fragrance. In certain embodiments of the compositions, these ingredients are present in the composition at about 0.5% w/w to about 20% w/w of the composition. In some embodiments, the composition comprises the one or more growth factors at about 20% w/w of the composition, disodium cocoamphodiacatate at about 10% w/w of the composition, and phenoxyethanol at about 0.5% w/w. In certain embodiments, the composition has a pH of about 6.3.

In another aspect, the disclosure provides a composition for cosmetic use, wherein the composition comprises ionized mineral water, one or more growth factors, a non-greasy emollient, polysorbate 20, vetiver essential oil, and phenoxyethanol. In certain embodiments of the compositions, these ingredients are present in the composition at about 0.5% w/w to about 20% w/w of the composition. In some embodiments, the composition comprises the one or more growth factors at about 20% w/w of the composition, vetiver essential oil at about 0.5% w/w of the composition, and phenoxyethanol at about 0.5% w/w of the composition. In certain embodiments, the composition has a pH of about 6.5.

In another aspect, the disclosure provides a composition for cosmetic use, wherein the composition comprises double distilled water, one or more growth factors, hyaluronic acid, anhydrous lanolin, lauryl glucoside, a non-greasy emollient, hydroxmethylcellulose, phenoxyethanol, and a fragrance. In certain embodiments of the compositions, these ingredients are present in the composition at about 0.5% w/w to about 30% w/w of the composition. In some embodiments, the composition comprises the one or more growth factors at about 30% w/w of the composition, hyaluronic acid at about 3% w/w of the composition, and phenoxyethanol at about 0.5% w/w of the composition. In certain embodiments, the composition has a pH of about 6.5.

In another aspect, the disclosure provides a composition for cosmetic use, wherein the composition comprises double distilled water, one or more growth factors, synergy organic oils of Olea europaea, argania spinosa, vitamin E, portulaca oleracea DNA, phenoxyethanol, and a fragrance. In certain embodiments of the compositions, these ingredients are present in the composition at about 0.5% w/w to about 20% w/w of the composition. In some embodiments, the composition comprises the one or more growth factors at about 20% w/w of the composition, argania spinosa at about 5% w/w of the composition, vitamin E at about 1% w/w of the composition, and phenoxyethanol at about 0.5% w/w of the composition. In certain embodiments, the composition has a pH of about 6.5.

In another aspect, the disclosure provides a composition for cosmetic use, wherein the composition comprises oat extract, one or more growth factors, fatty emollients emulsified with water, collagen, elastin, vitamin E, phenoxyethanol, and a fragrance. In certain embodiments of the compositions, these ingredients are present in the composition at about 0.5% w/w to about 30% w/w of the composition. In some embodiments, the composition comprises oat extract at 30% w/w of the composition, the one or more growth factors at about 20% w/w of the composition, collagen at about 2% w/w of the composition, elastin at about 2% w/w of the composition, and phenoxyethanol at about 0.5% w/w of the composition. In certain embodiments, the composition has a pH of about 6.5.

In another aspect, the disclosure provides a composition for cosmetic use, wherein the composition comprises double distilled water, one or more growth factors, mixture of fatty emollients emulsified with water, organic roseship oil, vitamin E, phenoxyethanol, and a fragrance. In certain embodiments of the compositions, these ingredients are present in the composition at about 0.5% w/w to about 20% w/w of the composition. In some embodiments, the composition comprises the one or more growth factors at about 20% w/w of the composition, organic roseship oil at about 3% w/w of the composition, and phenoxyethanol at about 0.5% w/w of the composition. In some embodiments, the composition has a pH of about 6.5.

In another aspect, the disclosure provides a composition for cosmetic use, wherein the composition comprises double distilled water, aloe vera extract, chamomile, one or more growth factors, ethoxylated lanolin, carbomer, triehtanolamine, collagen, elastin, portulaca oleracea DNA, phenoxyethanol, and a fragrance. In certain embodiments of the compositions, these ingredients are present in the composition at about 0.5% w/w to about 20% w/w of the composition. In some embodiments, the composition comprises aloe vera extract at about 10% w/w of the composition, chamomile at about 10% w/w of the composition, the one or more growth factors at about 20% w/w of the composition, and phenoxyethanol at about 0.5% w/w of the composition. In certain embodiments, the composition has a pH of about 6.5.

In another aspect, the disclosure provides a composition for cosmetic use, wherein the composition comprises double distilled water, oat extract, fat water soluble emollients, 2-ethyl hexyl salicylate, 2-ethyl hexyl 4-methoxycinnamate, 2-hydroxy-4-methoxybenzophenone, one or more growth factors, micronized titanium dioxide, phenoxyethanol, and a fragrance. In certain embodiments of the compositions, these ingredients are present in the composition at about 0.5% w/w to about 40% w/w of the composition. In some embodiments, the composition comprises oat extract at about 40% w/w of the composition, the one or more growth factors at about 5% w/w of the composition, and phenoxyethanol at about 0.5% w/w of the composition. In certain embodiments, the composition has a pH of about 6.5.

In another aspect, the disclosure provides a composition for cosmetic use, wherein the composition comprises distilled water, cell growth factors, mimosa tenuiflora extract, panthenol, shea butter, glycerin, propyleneglycol, xanthan gum, rosa rubiginosa essential oil, hyaluronic acid, aloe extract, and phenoxyethanol. In certain embodiments of the compositions, the ingredients other than the water are present in the composition at about 1% w/w to about 20% w/w of the composition. In some embodiments, the composition comprises distilled water at about 60.5% w/w of the composition, cell growth factors at about 20% w/w of the composition, mimosa tenuiflora extract at about 5% w/w of the composition, panthenol at about 3% w/w of the composition, shea butter at about 2% w/w of the composition, glycerin at about 1.5% w/w of the composition, propyleneglycol at about 1.5% w/w of the composition, xanthan gum at about 1.5% w/w of the composition, rosa rubiginosa at about 1% w/w of the composition, hyaluronic acid at about 1% w/w of the composition, aloe extract at about 2% w/w of the composition, and phenoxyethanol at about 1% w/w of the composition.

In another aspect, the disclosure provides a method of producing a composition for cosmetic or therapeutic use. The steps may include a) culturing mesenchymal cells (MSCs) in a suitable culture medium and under suitable culture conditions for at least about 72 hours from the time of collection up until about 3 months from the time of collection; b) centrifuging the culture of MSCs; c) carefully removing the supernatant from the centrifuged culture of MSCs; d) using flow cytometry to obtain a fraction of the supernatant that comprises growth factors, exosomes, and fragments of MSCs; and e) adding pharmaceutically acceptable excipients to the fraction, thereby producing the composition for cosmetic or therapeutic use. In some embodiments, the flow cytometry step comprises using fluorescence emission filters, dichroic filters, or both to obtain the desired fraction. The disclosure also provides a composition produced by this method. In some embodiments, additional ingredients are added to the composition. In some embodiments, any composition of the disclosure may be formulated as a gel, cream, lotion, serum, spray, patch, or solution.

In another aspect, the disclosure provides a method of treating an individual in need thereof by administering an effective amount of any of the compositions disclosed herein to the individual. In some embodiments, the composition is formulated as a gel, cream, lotion, serum, spray, patch, or solution. In some embodiments, the composition is administered topically. In other embodiments the composition is administered intradermally. In certain embodiments, the individual is in need of skin revitalization. In certain embodiments, the effective amount promotes hydration of the skin, stimulates production of collagen, increases elasticity of the skin, regulates the amount of skin fat, neutralizes free radicals, acts as a decongestant, decreases inflammation, or a combination thereof. In certain embodiments, the individual is in need of solar protection. In other embodiments, the composition is administered to an individual that has a wound, burn, or other injury.

### BRIEF DESCRIPTION OF THE FIGURES

FIG. 1 is an exemplary flow cytometry image, showing the presence of exosomes in a sample of MSC conditioned media.

FIG. 2 is an exemplary flow cytometry image, showing the epitopes present on exosomes in a sample of MSC conditioned media.

FIGS. 3A-3D are tables providing exemplary efficacy data from a twelve week study in which the disclosed compositions were used for the treatment of skin.

### DETAILED DESCRIPTION OF THE INVENTION

The following description recites various aspects and embodiments of the disclosed compositions and methods. No particular embodiment is intended to define the scope of the compositions and methods. Rather, the embodiments merely provide non-limiting examples that are at least included within the scope of the disclosed compositions and methods. The description is to be read from the perspective of one of ordinary skill in the art; therefore, information well known to the skilled artisan is not necessarily included.

### I. Introduction

Compositions are described herein for the treatment of skin. These novel compositions include various ingredients, such as growth factors and other advantageous components from stem cell or mesenchymal stem cell (MSC) cultures. Mesenchymal stem cells (MSCs) are adult stem cells and are a heterogeneous population of unspecialized cells capable of renewing themselves and differentiating into different meso, endo, or ectodermal cell lines under special culture conditions (Wei et al., 2013, Acta Pharmacologica Sinica, 34:747-754). A potential for transdifferentiation of MSCs to other cell lineages has been shown (e.g., mainly bone, cartilage, adipose tissue, muscle, supporting nervous, epithelial, pancreatic, hepatic and vascular tissue) (Lavoie & Rosu-Myles, 2013, J. Neurol. Sci., 324:1-9). MSCs are characterized by their ability to constantly divide for long periods of time without significant changes in their phenotype or general properties (Wei et al., 2013, Acta Pharmacologica Sinica, 34:747-754; Ding et al., 2011, Cell Transplantation, 20:5-14). Mesenchymal stem cells (MSCs) are multipotent cells derived from a variety of tissues including bone marrow, adipose tissue, placenta, endometrium, and umbilical cord blood. MSCs appear to have pleiotropic effects at the molecular level in all body cells, since they exert this effect via the paracrine pathway through the mediation of growth factors. Possible effects include migration to the site of damage (biodistribution towards areas of inflammation), immunomodulation, anti-inflammation (inhibition of local and systemic inflammatory response), hematopoietic cell support, antifibrosis, chemoattraction, angiogenesis, promotion of cell proliferation, prevention of tissue damage, control of oxidative stress, regulation of the cellular microenvironment (Spees et al., 2016, Stem Cell Research & Therapy, 7:125; Watt et al., 2013, Br. Med. Bull., 108:25-53). Since the discovery of MSCs over four decades ago, approximately 6,000 patients worldwide have been treated for different pathologies with cell therapy based on MSCs. Approximately 92% of the studies carried out to date correspond to the clinical study phase I and II (safety and efficacy), where an attempt has been made to discover MSCs-disease interactions, as well as their effectiveness and safety. There are currently ongoing studies focused on the use of MSCs for the treatment of various categories of conditions such as: mental and behavioral diseases; digestive system diseases; autoimmune diseases; ear, nose, and throat diseases; muscle, bone, and cartilage diseases; heart and blood diseases; respiratory tract diseases; urinary tract conditions; sexual organs and reproduction, among others (see, e.g., various studies described at https://clinicaltrials.gov). The compositions are stable and overcome the need for a cold chain network and lyophilization.

### II. Definitions

As used in herein, the singular forms “a,” “an,” and “the” include plural forms unless the content clearly dictates otherwise. Thus, for example, reference to “a growth factor” optionally includes a combination of two or more such molecules, and the like.

The term “exosome” refers to a small membrane-bound extracellular vesicle that is produced in the endosomal compartment of most eukaryotic cells. The invagination of the membrane of late endosomes, or multi-vesicular bodies, forms intraluminal vesicles within the multi-vesicular bodies. When the multi-vesicular bodies fuse with the plasma membrane, the intraluminal vesicles are released into the extracellular space as exosomes. The exosomes are functional membrane-bound vesicles that carry proteins, lipids, and nucleic acids of the cell from which they are derived, and they may deliver these molecules to other cells they encounter. In some embodiments, the exosomes are present in the disclosed compositions at a concentration of about 35 to about 75 exosomes/µL, about 45 to about 65 exsosomes/µL, or about 50 to about 60 exosomes/µL. In certain embodiments, the exosomes are present at a concentration of about 55 exosomes/µL.

The term “growth factor” refers to a naturally occurring biomolecule that acts as a signaling molecule and may stimulate cell proliferation, wound healing, and/or cellular differentiation. Examples of growth factors include, but are not limited to, cytokines and hormones. In some embodiments, growth factors may include VEGF, FGF, IL-6, CTGF, PDGF, PGE2, IL-10, HLA-G5, galectin 1, LIF, MHCII, EGF, TGF-β, IGF-1, KGF, SDF-1, MIP-1a, MIP-1b, SDF-1, or HGF, or a combination thereof. In some embodiments, the one or more growth factors comprise an Epithelial growth factor (EGF). In certain embodiments, the EGF is a human EGF. In some embodiments, the one or more growth factors comprise one or more Fibroblast growth factors (FGFs). In some embodiments, the one or more FGFs comprise a basic Fibroblast growth factor (bFGF). In certain embodiments, the bFGF is a human bFGF. In some embodiments, the one or more growth factors comprise a Platelet Derived Growth Factor (PDGF). In certain embodiments, the PDGF is a human PDGF. In some embodiments, the one or more growth factors comprise a Hepatocyte Growth Factor (HGF). In certain embodiments, the HGF is a human HGF. In some embodiments, the one or more growth factors comprise a growth differentiation factor-11 (GDF-11). In certain embodiments, the GDF-11 is a human GDF-11. In some embodiments, the one or more growth factors comprise a Transforming Growth Factor-beta (TGF-β). In certain embodiments, the TGF-β is a human TGF-β. In some embodiments, the one or more growth factors comprise Interleukin 6 (IL-6). In certain embodiments, the IL-6 is a human IL-6. In some embodiments, the one or more growth factors comprise Interleukin 10 (IL-10). In certain embodiments, the IL-10 is a human IL-10. In some embodiments, the one or more growth factors comprise a Chemokine (C-C motif) ligand 3 (CCL3; also referred to as macrophage inflammatory protein-1 α (MIP-1 α)). In certain embodiments, the CCL3 is a human CCL3. In some embodiments, the one or more growth factors comprise a Vascular Endothelial Growth Factor (VEGF). In certain embodiments, the VEGF is a human VEGF. In some embodiments, the one or more growth factors comprise a Leukemia Inhibitory Factor (LIF). In certain embodiments, the LIF is a human LIF. In some embodiments, the one or more growth factors comprise a Chemokine (C-C motif) ligand 4 (CCL4; also referred to as macrophage inflammatory protein-1 β (MIP-1 β)). In certain embodiments, the CCL4 is a human CCL4. In some embodiments of the disclosed compositions and methods, the one or more growth factors are one or more recombinant growth factors. In some embodiments of the disclosed compositions described further in the Examples, the term “growth factors” or “cell growth factors” may refer to one or more growth factors as described above or may refer to MSC conditioned media that includes one or more growth factors as described above.

As used herein, the term “DMDM hidandtoin” refers to a preservative for use in some embodiments of the disclosed compositions. DMDM hidantoin is an antimicrobial formaldehyde releaser with the formula C₇H₁₂N₂O₄.

As used here, the term “fragments of MSCs” refers to any noncellular portion of a MSC present in a culture of MSCs. For example, a fragment of MSC may include a MSC membrane or portion thereof.

The term “DNA” or “deoxyribonucleic acid” refers to a polymer of at least two or more nucleotides (e.g., adenine, thymine, cytosine, guanine). As used herein, the terms may encompass single stranded or double stranded forms of the nucleic acid.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The terms “individual,” “patient,” or “subject” are used interchangeably herein to refer to a patient in need of the particular described treatment. Preferably, “individual,” “patient,” or “subject” refers to a human.

The term “emollient” is used herein to refer to an ingredient that has smoothing or softening properties. A “water soluble emollient” refers to emollients that are soluble in water, including, but not limited to glycerin, sorbitol, and propylene glycol.

The terms “therapeutically effective dose,” “effective dose,” “effective amount,” or “therapeutically effective amount” herein is meant a dose that produces an effect for which it is administered. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of therapeutic effect of at least 5%, at least 10%, at least 15%, at least 20%, at least 25%, at least 40%, at least 50%, at least 60%, at least 75%, at least 80%, at least 90%, or at least 100% over a pre-treatment condition or control. Therapeutic efficacy can also be expressed as “-fold” increase or decrease. For example, a therapeutically effective amount can have at least 1.2-fold, at least 1.5-fold, at least 2-fold, at least 5-fold, or more effect over a control.

As used herein, the term “pharmaceutically acceptable excipient” refers to an excipient or diluent in a pharmaceutical composition (e.g., a substance other than the active ingredient in the composition). The pharmaceutically acceptable excipient must be compatible with the other ingredients of the formulation and not deleterious to the recipient at the dosages of concentrations employed. In some embodiments, the pharmaceutically acceptable excipient must provide adequate pharmaceutical stability to the active ingredient. Examples of pharmaceutically acceptable excipients include, but are not limited to, preservatives, coloring, fragrances, adjuvants, binders, and vehicles. The nature of the excipient may differ with the mode of administration.

Topical formulations may comprise certain types of excipients (e.g., polymers to control viscosity of the composition, surface active agents to help solubilize the active ingredients in the composition, preservatives to improve shelf life and microbiological stability of the composition, and penetration enhancers to promote absorption of the active ingredients in the composition).

In certain embodiments, the disclosed compositions can contain materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta- cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (See Allen (2012) Remington - The Science and Practice of Pharmacy, 22d Edition, Lloyd V, Allen, ed., The Pharmaceutical Press). In certain embodiments, the optimal compositions are determined by one skilled in the art depending upon, for example, the intended route of administration, delivery format, and desired dosage.

In certain embodiments, the primary vehicle or carrier in a disclosed composition for therapeutic use can be either aqueous or non-aqueous in nature. For example, in certain embodiments, a suitable vehicle or carrier can be water for injection or physiological saline solution for parenteral administration. In certain embodiments, the saline comprises isotonic phosphate-buffered saline. In certain embodiments, neutral buffered saline or saline mixed with serum albumin are further exemplary vehicles. In certain embodiments, the pharmaceutical compositions comprise Tris buffer of about pH 7.0-8.5, or acetate buffer of about pH 4.0-5.5, which can further include sorbitol or a suitable substitute therefore.

The term “treat” and “treatment” are used herein to refer to both therapeutic treatment and prophylactic or preventive measures, wherein the object is to prevent or slow down an undesired physiological change or disorder. For purpose of this disclosure, beneficial or desired results include, but are not limited to, decreasing tissue loss, promoting cell differentiation or tissue regeneration, alleviation of symptoms, diminishment of extent of symptoms, stabilized (i.e., not worsening) symptoms, or delay or slowing of symptom progression. “Treatment” can also refer to any detectable change or increase in quantity of a parameter that reflects a beneficial effect of the disclosed compositions, compared to a standard value determined in the absence of exposure to the disclosed compositions. The level of this change or increase in quantity of the parameter that reflects a beneficial effect following exposure to a composition as described herein is, in some embodiments, at least 5%, at least 10%, at least 20%, at least 30%, at least 40%, at least 50%, at least 60%, at least 70%, at least 80%, at least 90%, or 100%.

### III. Compositions for the Cosmetic or Therapeutic Treatment of Skin

Compositions are provided that include MSC conditioned media having components therein that are beneficial to the skin, including exosomes, one or more growth factors, and fragments of MSCs. Compositions are provided that include exosomes, one or more growth factors, optionally DMDM hidantoin, optionally phenoxyethanol, optionally benzyl alcohol, and fragments of mesenchymal stem cells (MSCs). In one aspect, the disclosure provides a composition for cosmetic or therapeutic use that includes exosomes, one or more growth factors, DMDM hidantoin, phenoxyethanol, benzyl alcohol, and fragments of MSCs. In certain embodiments of the compositions, these ingredients are present in the composition at about 0.1% w/w to about 40% w/w of the composition. In some embodiments, the composition comprises DMDM hidantoin at about 0.2% w/w, phenoxyethanol at about 0.3% w/w, and benzyl alcohol at about 0.3% w/w. In some embodiments, the exosomes are present at a concentration of about 55 exosomes/µL. In some embodiments, the growth factors comprise VEGF, FGF, IL-6, CTGF, PDGF, PGE2, IL-10, HLA-G5, galectin 1, LIF, MHCII, EGF, TGF-β, IGF-1, KGF, SDF-1, MIP-1a, MIP-1b, SDF-1, or HGF, or a combination thereof. In certain embodiments, the compositions are stable and overcome the need for a cold chain network and lyophilization.

In some embodiments, the disclosed compositions comprise one or more additional ingredients. In some embodiments, the disclosed compositions for cosmetic or therapeutic use comprise double distilled water, one or more growth factors, placenta hydrolyzate, plant DNA, a water soluble emollient, cellulose, phenoxyethanol, and a fragrance. In certain embodiments of the compositions, these ingredients are present in the composition at about 0.25% w/w to about 40% w/w of the composition, about 0.3% w/w to about 35% w/w of the composition, about 0.4% w/w to about 30% w/w of the composition, about 0.45% w/w to about 25% w/w of the composition, or between about 0.5% w/w to about 20% w/w of the composition. In some embodiments, the composition comprises the one or more growth factors at about 20% w/w of the composition, placenta hydrolyzate at about 5% w/w of the composition, plant DNA at about 3% w/w of the composition, and phenoxyethanol at about 0.5% w/w of the composition. In certain embodiments, the plant DNA is portulaca oleracea DNA. In certain embodiments, the composition has a pH of about 6.

In some embodiments of the compositions, the growth factors are present in the composition at about 2.5% w/w to about 40% w/w of the composition, about 5% w/w to about 35% w/w of the composition, about 5% w/w to about 30% w/w of the composition, about 7.5% w/w to about 25% w/w of the composition, or between about 10% w/w to about 20% w/w of the composition. In certain embodiments, growth factors are present in the composition at about 5% w/w of the composition. In certain embodiments, growth factors are present in the composition at about 10% w/w of the composition. In certain embodiments, growth factors are present in the composition at about 20% w/w of the composition. In certain embodiments, growth factors are present in the composition at about 30% w/w of the composition.

In certain embodiments of the compositions, placenta hydrolyzate is present in the composition at about 2% w/w to about 15% w/w of the composition, about 2.5% w/w to about 10% w/w of the composition, about 3% w/w to about 7.5% w/w of the composition, about 3.5% w/w to about 6% w/w of the composition, or between about 4% w/w to about 5% w/w of the composition. In certain embodiments, placenta hydrolyzate is present in the composition at about 5% w/w of the composition.

In certain embodiments of the compositions, plant DNA is present in the composition at about 0.25% w/w to about 15% w/w of the composition, about 0.5% w/w to about 10% w/w of the composition, about 1% w/w to about 7.5% w/w of the composition, about 1.5% w/w to about 4% w/w of the composition, or between about 2% w/w to about 3% w/w of the composition. In certain embodiments, plant DNA is present in the composition at about 3% w/w of the composition. In certain embodiments, the plant DNA is portulaca oleracea DNA.

In certain embodiments of the compositions, phenoxyethanol is present in the composition at about 0.2% w/w to about 5% w/w of the composition, about 0.25% w/w to about 3% w/w of the composition, about 0.3% w/w to about 2% w/w of the composition, about 0.35% w/w to about 1% w/w of the composition, or between about 0.4% w/w to about 0.5% w/w of the composition. In certain embodiments, phenoxyethanol is present in the composition at about 0.5% w/w of the composition.

In some embodiments, the disclosed compositions comprise one or more additional ingredients such as ionized mineral water, portulaca oleracea extract, a water soluble emollient, an exopolysaccharide, collagen, sodium L-Pyrrolidonecarboxylate (Sodium PCA or PcaNa), cellulose, elastin, thriethanolamine, vegetable glycerin, polysorbate 20, collagen, lauryl glucoside, disodium cocoamphodiacetate, ethoxylated lanonin, a non-greasy emollient, vetiver essential oil, hyaluronic acid, anhydrous lanolin, hydroxmethylcellulose, synergy organic oils of Olea europaea, argania spinosa, vitamin E, oat extract, fatty emollients emulsified with water, organic roseship oil, chamomile, carbomer, triethanolamine, 2-ethyl hexyl salicylate, 2-ethyl hexyl 4-methoxycinnamate, 2-hydroxy-4-methoxybenzophenone, micronized titanium dioxide, mimosa tenuiflora extract, panthenol, shea butter, glycerine, propyleneglycol, xanthan gum, aloe extract, or a fragrance. In certain embodiments of the compositions, these one or more additional ingredients are present in the composition at about 0.1% w/w to about 60% w/w of the composition, about 0.2% w/w to about 55% w/w of the composition, about 0.3% w/w to about 50% w/w of the composition, about 0.4% w/w to about 45% w/w of the composition, or between about 0.5% w/w to about 40% w/w of the composition.

In some embodiments of the compositions, portulaca oleracea extract is present in the composition at about 5% w/w to about 20% w/w of the composition, about 6% w/w to about 17.5% w/w of the composition, about 7% w/w to about 15% w/w of the composition, about 7.5% w/w to about 12.5% w/w of the composition, or between about 7.5% w/w to about 10% w/w of the composition. In certain embodiments, portulaca oleracea extract is present in the composition at about 10% w/w of the composition.

In some embodiments of the compositions, the exopolysaccharide is produced by plankton marine microalgae. In certain embodiments, the plankton marine microalgae are isolated in France, cultivated in a bioreactor, and extracted and purified with a membrane in no solvents and is 100% pure. In some embodiments of the compositions, an exopolysaccharide is present in the composition at about 0.25% w/w to about 15% w/w of the composition, about 0.5% w/w to about 10% w/w of the composition, about 1% w/w to about 7.5% w/w of the composition, about 1.5% w/w to about 4% w/w of the composition, or between about 2% w/w to about 3% w/w of the composition. In certain embodiments, the exopolysaccharide is present in the composition at about 3% w/w of the composition.

In some embodiments of the compositions, collagen is present in the composition at about 0.25% w/w to about 15% w/w of the composition, about 0.5% w/w to about 10% w/w of the composition, about 0.75% w/w to about 5% w/w of the composition, or about 1% w/w to about 3% w/w of the composition. In certain embodiments, collagen is present in the composition at 1% w/w of the composition. In certain embodiments, collagen is present in the composition at 2% w/w of the composition. In certain embodiments, collagen is present in the composition at about 3% w/w of the composition.

In some embodiments of the compositions, elastin is present in the composition at about 0.25% w/w to about 10% w/w of the composition, about 0.5% w/w to about 7.5% w/w of the composition, about 0.75% w/w to about 4% w/w of the composition, or about 1% w/w to about 3% w/w of the composition. In certain embodiments, elastin is present in the composition at 1% w/w of the composition. In certain embodiments, elastin is present in the composition at about 2% w/w of the composition.

In some embodiments of the compositions, plant DNA is present in the composition at about 0.25% w/w to about 15% w/w of the composition, about 0.5% w/w to about 10% w/w of the composition, about 1% w/w to about 7.5% w/w of the composition, about 1.5% w/w to about 4% w/w of the composition, or between about 2% w/w to about 3% w/w of the composition. In certain embodiments, the plant DNA is present in the composition at about 3% w/w of the composition. In certain embodiments, the plant DNA is portulaca oleracea DNA.

In some embodiments of the compositions, Sodium PCA is present in the composition at about 0.25% w/w to about 15% w/w of the composition, about 0.5% w/w to about 10% w/w of the composition, about 1% w/w to about 7.5% w/w of the composition, about 1.5% w/w to about 4% w/w of the composition, or between about 2% w/w to about 3% w/w of the composition. In certain embodiments, the sodium PCA is present in the composition at about 3% w/w of the composition.

In some embodiments of the compositions, disodium cocoamphodiacatate is present in the composition at about 5% w/w to about 20% w/w of the composition, about 6% w/w to about 17.5% w/w of the composition, about 7% w/w to about 15% w/w of the composition, about 7.5% w/w to about 12.5% w/w of the composition, or between about 7.5% w/w to about 10% w/w of the composition. In certain embodiments, disodium cocoamphodiacatate is present in the composition at about 10% w/w of the composition.

In certain embodiments of the compositions, vetiver essential oil is present in the composition at about 0.2% w/w to about 5% w/w of the composition, about 0.25% w/w to about 3% w/w of the composition, about 0.3% w/w to about 2% w/w of the composition, about 0.35% w/w to about 1% w/w of the composition, or between about 0.4% w/w to about 0.5% w/w of the composition. In certain embodiments, vetiver essential oil is present in the composition at about 0.5% w/w of the composition.

In some embodiments of the compositions, hyaluronic acid is present in the composition at about 0.25% w/w to about 15% w/w of the composition, about 0.5% w/w to about 10% w/w of the composition, about 1% w/w to about 7.5% w/w of the composition, about 1.5% w/w to about 4% w/w of the composition, or between about 2% w/w to about 3% w/w of the composition. In certain embodiments, the hyaluronic acid is present at about 1% of w/w of the composition. In certain embodiments, the hyaluronic acid is present in the composition at about 3% w/w of the composition.

In certain embodiments of the compositions, argania spinosa is present in the composition at about 2% w/w to about 15% w/w of the composition, about 2.5% w/w to about 10% w/w of the composition, about 3% w/w to about 7.5% w/w of the composition, about 3.5% w/w to about 6% w/w of the composition, or between about 4% w/w to about 5% w/w of the composition. In certain embodiments, argania spinosa is present in the composition at about 5% w/w of the composition.

In some embodiments of the compositions, vitamin E is present in the composition at about 0.25% w/w to about 10% w/w of the composition, about 0.25% w/w to about 7.5% w/w of the composition, about 0.5% w/w to about 5% w/w of the composition, or about 1% w/w to about 3% w/w of the composition. In certain embodiments, vitamin E is present in the composition at about 1% w/w of the composition.

In some embodiments of the compositions, oat extract is present in the composition at about 5% w/w to about 60% w/w of the composition, about 10% w/w to about 55% w/w of the composition, about 15% w/w to about 50% w/w of the composition, about 20% w/w to about 45% w/w of the composition, or between about 30% w/w to about 40% w/w of the composition. In certain embodiments, oat extract is present in the composition at 30% w/w of the composition. In certain embodiments, oat extract is present in the composition at about 40% w/w of the composition.

In some embodiments of the compositions, organic roseship oil is present in the composition at about 0.25% w/w to about 15% w/w of the composition, about 0.5% w/w to about 10% w/w of the composition, about 1% w/w to about 7.5% w/w of the composition, about 1.5% w/w to about 4% w/w of the composition, or between about 2% w/w to about 3% w/w of the composition. In certain embodiments, the organic roseship oil is present in the composition at about 3% w/w of the composition. In certain embodiments, the roseship oil is present in the composition at about 1% w/w of the composition.

In some embodiments of the compositions, aloe vera extract is present in the composition at about 1% w/w to about 20% w/w of the composition, about 2% w/w to about 17.5% w/w of the composition, about 5% w/w to about 15% w/w of the composition, about 7.5% w/w to about 12.5% w/w of the composition, or between about 7.5% w/w to about 10% w/w of the composition. In certain embodiments, aloe vera extract is present in the composition at about 10% w/w of the composition. In certain embodiments, aloe extract is present in the composition at about 2% w/w of the composition.

In some embodiments of the compositions, chamomile is present in the composition at about 5% w/w to about 20% w/w of the composition, about 6% w/w to about 17.5% w/w of the composition, about 7% w/w to about 15% w/w of the composition, about 7.5% w/w to about 12.5% w/w of the composition, or between about 7.5% w/w to about 10% w/w of the composition. In certain embodiments, chamomile is present in the composition at about 10% w/w of the composition.

In some embodiments of the compositions, mimosa tenuiflora extract is present in the composition at about 0.5% w/w to about 20% w/w of the composition, about 1% w/w to about 15% w/w of the composition, about 2% w/w to about 10% w/w of the composition, about 3% w/w to about 7% w/w of the composition, or between about 4% w/w to about 6% w/w of the composition. In certain embodiments, mimosa tenuiflora extract is present in the composition at about 5% w/w of the composition.

In some embodiments of the compositions, panthenol is present in the composition at about 0.1% w/w to about 20% w/w of the composition, about 0.5% w/w to about 15% w/w of the composition, about 1% w/w to about 10% w/w of the composition, about 1.5% w/w to about 6% w/w of the composition, or between about 2% w/w to about 4% w/w of the composition. In certain embodiments, panthenol is present in the composition at about 3% w/w of the composition.

In some embodiments of the compositions, shea butter is present in the composition at about 0.1% w/w to about 20% w/w of the composition, about 0.2% w/w to about 10% w/w of the composition, about 0.5% w/w to about 7.5% w/w of the composition, about 1% w/w to about 6% w/w of the composition, or between about 1.5% w/w to about 3% w/w of the composition. In certain embodiments, shea butter is present in the composition at about 2% w/w of the composition.

### IV. Methods for the Cosmetic or Therapeutic Treatment of Skin

The present disclosure provides a method of treating an individual in need thereof by administering an effective amount of any of the compositions disclosed herein to the individual. In some aspects, the compositions are administered for cosmetic use. In some embodiments, the composition is formulated as a gel, cream, lotion, serum, spray, patch, or solution. In some embodiments, the composition is administered topically. In certain embodiments, the individual is in need of skin revitalization. In certain embodiments, the effective amount promotes hydration of the skin, stimulates production of collagen, increases elasticity of the skin, regulates the amount of skin fat, neutralizes free radicals, acts as a decongestant, decreases inflammation, or a combination thereof. In some embodiments, the effective amount of the composition comprises a concentration of about 5% to about 50% MSC conditioned medum, about 10% to about 40% MSC conditioned medum, or about about 15% to about 35% MSC conditioned medum. In certain embodiments, the effective amount is at a concentration of about 20% to about 30% MSC conditioned medum. In certain embodiments, the individual is in need of solar protection.

In some aspects, the compositions are administered for therapeutic use. In some embodiments, the composition is formulated as a gel, cream, lotion, serum, spray, patch, or solution. In some embodiments, the composition is administered locally by topical administration or injection. In certain embodiments, the individual is in need of damage repair and/or wound healing. In certain embodiments, the compositions cause migration to the site of damage (biodistribution towards areas of inflammation), immunomodulation, anti-inflammation (inhibition of local and systemic inflammatory response), hematopoietic cell support, antifibrosis, chemoattraction, angiogenesis, promotion of cell proliferation, prevention of tissue damage, or control of oxidative stress, or combinations thereof. In some embodiments, the effective amount of the composition comprises a concentration of about 5% to about 50% MSC conditioned medum. In other embodiments, the disclosed compositions may be used for the treatment of various categories of conditions that are being treated with use of MSCs, such as: mental and behavioral diseases; digestive system diseases; autoimmune diseases; ear, nose and throat diseases; muscle, bone and cartilage diseases; heart and blood diseases; respiratory tract diseases; urinary tract conditions; and sexual organs and reproduction.

### V. Pharmaceutical Compositions

The disclosed compositions for cosmetic or therapeutic use can be provided in a pharmaceutical composition. The pharmaceutical compositions may comprise a pharmaceutically acceptable carrier or excipient. Pharmaceutically acceptable carriers or excipients are determined in part by the particular composition being administered, as well as by the particular method used to administer the composition. Accordingly, there are a wide variety of suitable formulations of pharmaceutical compositions of the present disclosure (see, e.g., Remington’s Pharmaceutical Sciences, 17th ed., 1989).

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. The formulations of ingredients can be presented in unit-dose or multi-dose sealed containers, such as ampoules and vials.

In certain embodiments, the pharmaceutical composition can be selected for parenteral delivery, particularly intradermal injection. The preparation of such pharmaceutically acceptable compositions is within the ability of one skilled in the art. In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments when parenteral administration is contemplated, a therapeutic composition can be in the form of a pyrogen-free, parenterally acceptable aqueous solution comprising the active ingredients, in a pharmaceutically acceptable vehicle. In certain embodiments, a vehicle for parenteral injection is sterile distilled water in which the active ingredients is formulated as a sterile, isotonic solution, and properly preserved. In certain embodiments, the preparation can involve the formulation of the active ingredients with an agent, such as injectable microspheres, bio-erodible particles, polymeric compounds (such as polylactic acid or polyglycolic acid), beads, or liposomes, that can provide for the controlled or sustained release of the active ingredients which can then be delivered via a depot injection. In certain embodiments, hyaluronic acid can also be used, and can have the effect of promoting sustained duration of the active ingredients of the composition.

The dose administered to a patient should be sufficient to effect a beneficial response in the subject over time. The optimal dose level for any patient will depend on a variety of factors including the efficacy of the composition employed, the age, body weight, physical activity, and diet of the patient, and on a possible combination with other drugs. The dose also will be determined by the existence, nature, and extent of any adverse side-effects that accompany the administration of a particular composition in a particular subject.

In determining the effective amount of the composition to be administered, a physician may evaluate the beneficial effects of the composition as well as its side effects. In general, the skin’s absorption is selective, and the compositions will not cause toxicity as metabolism is more difficult through the skin barrier, which is like a sponge and localized. The amount of the composition administered to the skin or wound must be metabolized in the seventh layer of the skin.

In some embodiments, the disclosed compositions are used for treatment of the skin of an individual by topical administration of an effective amount of the disclosed compositions. The compositions for topically treating skin may be administered on a regular basis for a period of time (e.g., 2, 3, 4, 5, 6, days, months or a year or more). In some embodiments, the composition is administered to an individual an average of about once a day, two times a day, three times a day, or more than three times per day. In some embodiments, the composition is administered to an individual an average of about once or twice per day. In certain embodiments, the composition is administered an average of about twice per day, typically in the morning upon rising and in the evening before retiring.

### VI. Methods for Production of the Cosmetic or Therapeutic Compositions

Methods are provided for the production of a composition for cosmetic or therapeutic use. The steps may include a) culturing mesenchymal cells (MSCs) in a suitable culture medium and under suitable culture conditions for about 72 hours from the time of collection up until about 3 months from the time of collection; b) centrifuging the culture of MSCs; c) carefully removing the supernatant from the centrifuged culture of MSCs; d) using flow cytometry to obtain a fraction of the supernatant that comprises growth factors, exosomes, and fragments of MSCs; and e) adding pharmaceutically acceptable excipients to the fraction, thereby producing the composition for cosmetic or therapeutic use. In some embodiments, the flow cytometry step comprises using fluorescence emission filters, dichroic filters, or both to obtain the desired fraction. The disclosure also provides a composition produced by this method. In some embodiments, additional ingredients are added to the composition produced by this method. In some embodiments, any composition of the disclosure may be formulated as a gel, cream, lotion, serum, spray, patch, or solution.

### EXAMPLES

The following examples are offered to illustrate, but not to limit the claimed invention.

### Example 1 - Preparation of Stem Cell Culture Components

Though different protocols have been reported for the isolation, characterization, and expansion of MSCs, all reports identify the minimum criteria of MSCs as being cells that 1) show adherence to plastic surfaces, 2) possess a specific group of cell surface markers (e.g., presence of cluster of differentiation (CD)73, CD90, CD105) and absence of CD14, CD34, CD45, and human leukocyte antigen-DR (HLA-DR) markers, and 3) show the ability to differentiate, under in vitro conditions, into osteoblasts, chondrocytes, and adipocytes.

### Donor Selection

Human donors between 18 and 45 years of age and that are in good general health were selected (between 18 and 30 for placental tissue and umbilical cord donors). The donors had not had a bacterial infection or viral illness within the last year and no history of inherited family diseases. Donors were free of communicable diseases (e.g., HIV/AIDS, hepatitis B and C, syphilis, cytomegalovirus, and intracellular bacteria). Individuals with high blood pressure were potential donors if their blood pressure was well controlled and there was no evidence of associated cardiovascular comorbidities or complications. In addition, donors did not have diabetes mellitus, arthritis, asthma, epilepsy, heart disease, or an autoimmune disease (e.g., multiple sclerosis, systemic lupus erythematosus, fibromyalgia). Donors also had not had any type of cancer, hip replacement surgery or a hip fracture, an organ transplant, or pulmonary tuberculosis within the last two years. Donors of placental tissue and umbilical cord tissue also had not received hormone therapy to achieve pregnancy or had premature rupture of the membranes greater than 24 hours of evolution.

### Procedure for Tissue Extraction

For adipose tissue extraction, the periumbilical abdominal region of the donor was sterilized and treated with a local anesthetic (e.g., lidocaine). Klein’s solution was inserted with a cannula into the orifices in the mid-deep subcutaneous space. After 20 minutes, a suction cannula was used to extract about 40 to about 60 mL of adipose tissue. About 40 mL adipose tissue was equivalent to about 40 g of fat and was suitable for the isolation of a sufficient number of stem cells. The containers of tissue were placed in airtight containers, labeled, and transported with cooling gels to maintain the tissue at about 4° C. to about 8° C. until processing. In some instances, the tissue samples were processed immediately after extraction.

For endometrial tissue extraction, the areas of cervix and fornix of the donor were sterilized and treated with a local anesthetic. A biopsy cannula was used to aspirate the endometrial tissue. The tissue was maintained at about 4° C. to about 8° C. until processing. In some instances, the tissue samples were processed immediately after extraction.

For menstrual tissue extraction, the donor was provided a menstrual cup to use on the day of donation. Sterile gloves were used to remove the cup and collect the sample to avoid contamination. The tissue was maintained at about 4° C. to about 8° C. until processing. In some instances, the tissue samples were processed immediately after extraction.

For placental or umbilical cord donation, when the donor was undergoing a caesarean section, the placental or umbilical cord tissue was obtained and placed in an a sterile bag or other receptacle in which antibiotics and water were added to maintain sterility. The bag or receptacle was transported in an airtight container with cooling gels to maintain the tissue at about 4° C. to about 8° C. until processing. In some instances, the tissue samples were processed immediately after extraction.

### MSC Extraction and Culture

MSCs were extracted from the adipose, endometrial, menstrual, or placental or umbilical cord tissue according to procedures known in the art (See, e.g., Kim et al., 2020, Biomed. & Pharmacother., 131, 110789; Kim et al., 2018, Biochem. Biophys. Rep., 16:96-102; Zhou et al., 2013, Biomed. Res. Int′l., 519126; Xu et al., 2016, Stem Cells Int′l., 7315830; Agrawal et al., 2011, Tissue Eng., 17(19-20):2435-2443; Reing et al., 2010, Biomater., 31(33):8626-8633). Extracted MSCs were then cultured under conditions suitable for the maintenance and cultivation of the MSCs (See, e.g., Kim et al., 2020, Biomed. & Pharmacother., 131, 110789; Kim et al., 2018, Biochem. Biophys. Rep., 16:96-102; Zhou et al., 2013, Biomed. Res. Int′l., 519126; Xu et al., 2016, Stem Cells Int′l., 7315830; Agrawal et al., 2011, Tissue Eng., 17(19-20):2435-2443).

### Preparation of MSC Conditioned Medium

MSCs were cultured in a suitable culture medium and under suitable culture conditions after collection. In some embodiments, the MSCs are cultured in DMEM low glucose medium (Dulbecco’s Modified Eagle’s Medium). The culture medium was obtained from the culture after 72 hours from the time of collection, up until about 3 months from the time of collection, by centrifugation. Flow cytometry was used to obtain a fraction of the supernatant that comprises growth factors, exosomes, and fragments of MSCs. This fraction was analyzed further as described in the following examples.

### Example 2 - Analysis of MSC Conditioned Medium

### Flow Cytometry for Exosome Detection

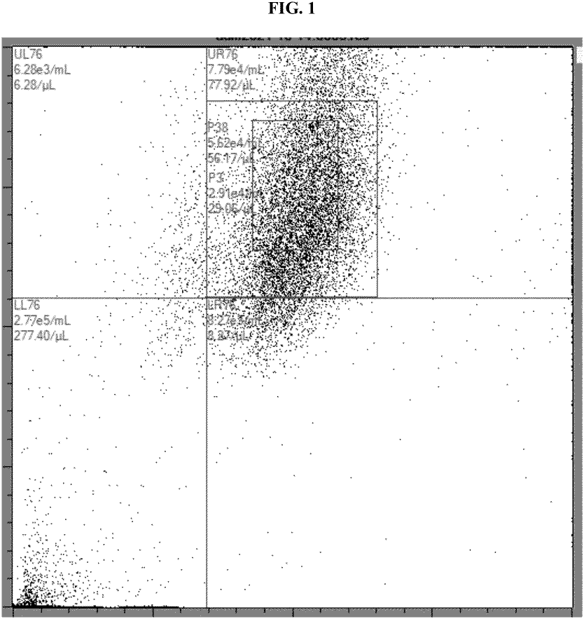

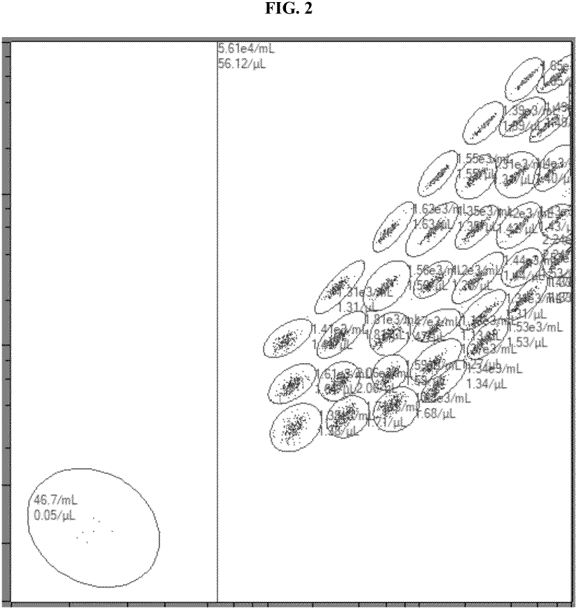

Samples of the MSC conditioned media were used in flow cytometry analysis to determine the concentration of exosomes in the sample. The analysis used markers with specific fluorochromes (CD3, CD4, CD19, CD8, HLA-DRDPDQ, CD56, CD105, CD2, CD1c, CD25, CD49e, ROR1, CD209, CD9, SSEA-4, HLA-ABC, CD63, CD40, CD62-P, CD11c, CD81, MCSP, CD146, CD41b, CD42a, CD24, CD86, CD44, C326, CD133/1, CD29, CD69, CD142, CD45, REA Control, CD20, CD14, mIgG1). Certain markers are specifically enriched in the membrane of exosomes (e.g., CD9, CD63, CD81) (See, e.g., Andreu & Yanez-Mo, 2014, Front. Immunol., 5:42). Therefore, antibodies to CD9, CD63, and CD81 were first used to identify the region positive for exosomes in the sample (FIG. 1). The selection of the population for analysis was made with respect to graph of size (forward scatter, FSC) and granularity (side scatter, SSC). Thirty-five antibodies used in the analysis fell in the specific region for exosomes and were selected for quantification of the exosomes (FIG. 2). Based on this analysis, it was determined that the sample includes 56.17 exosomes/µL of sample.

### Enzyme-Linked Immunoassay for Growth Factor Detection

Multiplex Enzyme-Linked Immunoassays (ELISAs) were performed in triplicate using a Luminex MAGPIX system according to the manufacturer’s recommended conditions to confirm the presence or absence of certain growth factors in the MSC cultured media and in several disclosed compositions that incorporate the MSC cultured media. The results are shown in Table 1 below. P.C. refers to the positive control for each of the growth factors that were tested in the samples. The positive controls for each of the growth factors was properly diluted to be between the standard as shown on the Certificate of Analysis (data not shown). The concentrations detected in each of the samples are listed in the table (pg/mL), and growth factors that were detected in a sample at a concentration less than 50 pg/mL are shown as “Undetermined.”

TABLE 1

| Results (pg/mL) | P.C. Active ingredient | Repair Serum | Regenerative Serum | Hidratonic | Facial Cleanser |
|---|---|---|---|---|---|
| IL-6 | 33462.69 | 2196.06 | 3721.51 | 1434.60 | 50.03 |
| IL-10 | 31516.96 | 2002.53 | 2989.07 | Undetermined | 93.65 |
| CCL3 MIP | 577662.47 | 59709.83 | 72740.82 | 21298.51 | 6703.49 |
| FGF | 20518.54 | 1253.25 | 3272.22 | 86.80 | Undetermined |
| HGF | 170588.77 | 16760.54 | 28075.35 | 268.87 | Undetermined |
| PDGF | 37487.73 | 2724.38 | 4271.75 | 193.87 | 85.41 |
| VEGF | 56852.83 | 4186.29 | 6923.86 | 195.77 | Undetermined |
| EGF | 90084.28 | 8898.42 | 12342.73 | Undetermined | Undetermined |
| LIF | 280546.70 | 22606.50 | 39975.09 | Undetermined | undetermined |
| CCL4 MIP | 873360.47 | 64087.06 | 94619.34 | Undetermined | Undetermined |

### Example 3 - EXODERM

Skin care compositions are provided for the treatment of skin. In some embodiments, the compositions comprise mesenchymal stem cell (MSC) conditioned medium, including exosomes, one or more growth factors, and fragments of MSCs. In some embodiments, the compositions for cosmetic or therapeutic use include exosomes, one or more growth factors, DMDM hidantoin, phenoxyethanol, benzyl alcohol, and fragments of MSCs. In certain embodiments of the compositions, these ingredients are present in the composition at about 0.2% w/w to about 40% w/w of the composition. In some embodiments, the composition comprises DMDM hidantoin at 0.2%, phenoxyethanol at 0.3%, and benzyl alcohol at 0.3%. In certain embodiments, the exosomes are present at a concentration of about 55 exosomes/µL. In some embodiments, the growth factors comprise VEGF, FGF, IL-6, CTGF, PDGF, PGE2, IL-10, HLA-G5, galectin 1, LIF, NMCII, EGF, TGF-β, IGF-1, KGF, SDF-1, MIP-1a, MIP-1b, SDF-1, or HGF, or a combination thereof. The compositions are stable and overcome the need for a cold chain network and lyophilization.

### Example 4 - Revitalizing Facial Serum

Skin care compositions are provided for the treatment of sensitive or devitalized skin. In some embodiments, the compositions include Double Distilled Water, Cell Growth Factors, Placenta Hydrolyzate, Portulaca oleracea DNA, Water-Soluble Emollients, Cellulose, Phenoxyethanol, and Fragrance/Aroma. In certain embodiments, the compositions include some of these components at the concentrations indicated in Table 2, with the other components making up the remainder of the composition. The compositions are for external use only.

TABLE 2

| | |
|---|---|
| Component | % w/w |
| Cell Growth Factors | 20% w/w |
| Placenta Hydrolyzate | 5% w/w |
| Portulaca oleracea DNA | 3% w/w |
| Phenoxyethanol | 0.5% w/w |

The compositions are used at home or administered by a professional for the treatment of sensitive skin and/or for a lifting effect. A portion of the composition is applied to clean and toned skin of the neck and face with circular movements until it has been completely absorbed. The composition is applied twice daily, in the morning and night. This treatment results in the rebuilding of the skin matrix, generating a delicate film with a lifting effect. The composition provides a deep hydration, instantly improving the appearance and relief of the skin.

### Example 5 - Eye Contour Lifting Cream

Skin care compositions are provided for treatment of sensitive or devitalized skin in the area around the eye. In some embodiments, the compositions include Double Distilled Water, Cell Growth, Portulaca oleracea fluid extract, Water-Soluble Emollients, ExoPolySaccharide, Collagen, sodium PCA, Cellulose, Elastin, Thriethanolamine, Phenoxythanol, and Fragrance/ Aroma. In certain embodiments, the compositions include some of these components at the concentrations indicated in Table 3, with the other components making up the remainder of the composition. The compositions are for external use only.

TABLE 3

| | |
|---|---|
| Component | % w/w |
| Cell Growth Factors | 20% w/w |
| Portulaca oleracea fluid extract | 10% w/w |
| ExoPolySaccharide | 3% w/w |
| Collagen | 1% w/w |
| Elastin | 1% w/w |
| Phenoxyethanol | 0.5% w/w |

The compositions are used at home or administered by a professional for the treatment of sensitive skin and/or for a lifting effect around the eyes. A portion of the composition is applied to clean and toned skin around the eye contour in circular movements until it has been completely absorbed. The composition is applied twice daily, in the morning and night. This treatment results in stimulating effects on the skin around the eyes, with nourishing, anti-wrinkle, tensing, and activating actions of cell renewal. With a firming and moisturizing effect on the skin around the eyes, the composition stimulates the production of natural collagen and increases the elasticity of the skin around the eyes. The composition also regulates the amount of skin fat around the eyes.

### Example 6 - Hydrating Facial Toner

Skin care compositions are provided for treatment of sensitive or devitalized skin. In some embodiments, the compositions include Double Distilled Water, Cell Growth Factors, Portulaca oleracea fluid extract, Vegetable Glycerin, Polysorbate 20, Portulaca oleracea DNA, Phenoxythanol, Fragrance/Aroma. In certain embodiments, the compositions include some of these components at the concentrations indicated in Table 4, with the other components making up the remainder of the composition. The compositions are for external use only.

TABLE 4

| | |
|---|---|
| Component | % w/w |
| Cell Growth Factors | 10% w/w |
| Portulaca oleracea fluid extract | 10% w/w |
| Portulaca oleracea DNA | 3% w/w |
| Phenoxyethanol | 0.5% w/w |

The compositions are used at home or administered by a professional for the treatment of skin. After facial cleansing, the composition is deliberately sprayed on the surface of the neck and face. The composition is applied before and after the physical or mechanical exfoliation of the skin and helps cleanse the skin and removes impurities. The composition is activated by massaging it into the skin until it has been completely absorbed. The composition provides a hydrating and antioxidant effect and helps skin regeneration and moisturizing for a long period of time. Use of the composition results in an increase in blood flow in the area due to its toning effect.

### Example 7 - Facial Moisturizing Gel

Skin care compositions are provided for treatment of sensitive or devitalized skin. In some embodiments, the compositions include Double Distilled Water, Cell Growth Factors, Portulaca oleracea fluid extract, Water-Soluble Emollients, Portulaca oleracea DNA, Collagen, sodium PCA, Cellulose, Elastin, Phenoxythanol, and Fragrance/Aroma. In certain embodiments, the compositions include some of these components at the concentrations indicated in Table 5, with the other components making up the remainder of the composition. The compositions are for external use only.

TABLE 5

| | |
|---|---|
| Component | % w/w |
| Cell Growth Factors | .10% w/w |
| Collagen | 3% w/w |
| Elastin | 2% w/w |
| Sodium PCA | 3% w/w |
| Phenoxyethanol | 0.5% w/w |

The compositions are used at home or administered by a professional for the treatment of sensitive skin and/or for lifting effect. A portion of the gel is applied on the neck and face surface with circular movements until it has been completely absorbed. The composition is applied twice daily, in the morning and night. These compositions that include Cell Growth Factors and Plant DNA provide intense hydration. Use of the composition improves cell metabolism and softens and regenerates the elastic and collagen fibers of the skin tissue. The composition also has a neutralizing action on free radicals and acts as a decongestant and anti-inflammatory.

### Example 8 - Facial Cleanser

Skin care compositions are provided for grooming and skin activation. In some embodiments, the compositions include Double Distilled Water, Lauryl glucoside, Disodium cocoamphodiacatate, Ethoxylated lanonin, Cellulose, Phenoxythanol, and Fragrance/Aroma. In certain embodiments, the compositions include some of these components at the concentrations indicated in Table 6, with the other components making up the remainder of the composition. The compositions are for external use only.

TABLE 6

| | |
|---|---|
| Component | % w/w |
| Cell Growth Factors | 20% w/w |
| Disodium cocoamphodiacatate | 10% w/w |
| Phenoxyethanol | 0.5% w/w |

The compositions are used at home or administered by a professional for conditioning and preparation of the skin before any dermo-cosmetic of infiltration treatment. The facial foam is applied on the neck and face surface with circular movements and then rinsed with water. The composition is gentle enough to be applied twice daily, in the morning and night. These compositions safely remove makeup and impurities from the skin. Use of the composition cleans, calms, and relaxes the skin; maintains the natural balance of pH on the skin; and provides anti-aging benefits. The composition does not contain sulfates and parabens.

### Example 9 - Hydrating Facial Toner

Skin care compositions are provided for treatment of all skin types, particularly ideal for dehydrated or devitalized skin. In some embodiments, the compositions include Ionized mineral water, Cell Growth Factors, Non-greasy emollients, Polysorbate 20, Vetiver essential oil, and Phenoxythanol. In certain embodiments, the compositions include some of these components at the concentrations indicated in Table 7, with the other components making up the remainder of the composition. The compositions are for external use only.

TABLE 7

| | |
|---|---|
| Component | % w/w |
| Cell Growth Factors | 20% w/w |
| Vetiver | 0.5% w/w |
| Phenoxyethanol | 0.5% w/w |

The compositions are used at home or administered by a professional after facial cleansing. The compositions are deliberately sprayed on the surface of the neck and face before and/or after a physical or mechanical exfoliation. The compositions are activated by massaging until they are completely absorbed. Use of the compositions is ideal for mature skin and dehydrated or dry skin due to its soothing, regenerating, firming, revitalizing, and anti-aging properties. Formulated with cell growth factor and ionized mineral water, the compositions hydrate, repair, and soften the skin with great antioxidant firming power.

### Example 10 - Nourishing Facial Serum With Hyaluronic Acid

Skin care compositions are provided for rejuvenating mature skin or prematurely aging skin. In some embodiments, the compositions include Double Distilled Water, Cell Growth Factors, Hyaluronic Acid, Anhydrous lanolin Lauryl glucoside, Non-greasy emollients, Hydroxymethylcellulose, Phenoxythanol, and Fragrance/Aroma. In certain embodiments, the compositions include some of these components at the concentrations indicated in Table 8, with the other components making up the remainder of the composition. The compositions are for external use only.

TABLE 8

| | |
|---|---|
| Component | % w/w |
| Cell Growth Factors | 30% w/w |
| Hyaluronic Acid | 3% w/w |
| Phenoxyethanol | 0.5% w/w |

The compositions are used at home or administered by a professional for facial rejuvenation and are compatible for use with Microneedling techniques. The composition is applied over the surface of the neck and face with circular movements until it has been completely absorbed. In certain embodiments, the composition is applied cold twice daily, in the morning and at night. Use of the composition results in the hydration of the epidermis and helps to reconstitute the fibers that support the skin tissues. Use of the compositions also increases the skin’s firmness, improves elasticity of the skin, and stimulates cell renewal. The composition is used to combat the signs of skin aging, to act as an antioxidant, and to fight free radicals that cause skin deterioration. The composition is an effective moisturizer, is ideal for dry skin, and improves skin tone and keeps it young in appearance.

### Example 11 - Anti-Aging Facial Serum

Skin care compositions are provided for rejuvenating mature skin or prematurely aging skin. In some embodiments, the compositions include Double Distilled Water, Cell Growth Factors, Synergy organic oils of Olea europaea, Argania spinosa, Vitamin E, Portulaca oleracea DNA, Phenoxythanol, and Fragrance/Aroma. In certain embodiments, the compositions include some of these components at the concentrations indicated in Table 9, with the other components making up the remainder of the composition. The compositions are for external use only.

TABLE 9

| | |
|---|---|
| Component | % w/w |
| Cell Growth Factors | 0.20% w/w |
| Argania spinosa | 5% w/w |
| Vitamin E | 1% w/w |
| Phenoxyethanol | 0.5% w/w |

The compositions are used at home or administered by a professional for facial rejuvenation. The composition is applied twice daily (morning and night) over the surface of the neck and face with circular movements until it has been completely absorbed. Use of the composition contributes to the structural role of cells, providing firmness and elasticity to the skin, as well as improving the general condition of the skin. The composition is a powerful moisturizer that improves the conditions of the cell system, providing hydration with a revitalizing, moisturizing, firming, antioxidant, and anti-aging action.

### Example 12 - Moisturizing Facial Cream

Skin care compositions are provided for rejuvenating mature skin or prematurely aging skin. In some embodiments, the compositions include Oat Extract, Cell Growth Factors, Mixture of fatty emollients emulsified with water, Collagen, Elastin, Vitamin E, Phenoxythanol, and Fragrance/Aroma. In certain embodiments, the compositions include some of these components at the concentrations indicated in Table 10, with the other components making up the remainder of the composition. The compositions are for external use only.

TABLE 10

| | |
|---|---|
| Component | % w/w |
| Oat extract | 30% w/w |
| Cell Growth Factors | 20% w/w |
| Collagen | 2% w/w |
| Elastin | 2% w/w |
| Phenoxyethanol | 0.5% w/w |

The compositions are used at home or administered by a professional for facial rejuvenation. The composition is applied over the surface of the neck and face with circular movements until it has been completely absorbed. The composition is applied twice daily (morning and night). Use of this anti-aging cream with cell growth factors stimulates cell regeneration, reduces wrinkles, and recovers skin turgor. The compositions are enriched with collagen and elastin to achieve an even more beneficial effect.

### Example 13 - Nourishing Nighttime Facial Cream

Skin care compositions are provided for rejuvenating mature skin or prematurely aging skin. In some embodiments, the compositions include Double Distilled Water, Cell Growth Factors, Mixture of fatty emollients emulsified with water, Organic Rosehip Oil, Vitamin E, Phenoxythanol, and Fragrance/Aroma. In certain embodiments, the compositions include some of these components at the concentrations indicated in Table 11, with the other components making up the remainder of the composition. The compositions are for external use only.

TABLE 11

| | |
|---|---|
| Component | % w/w |
| Cell Growth Factors | 20% w/w |
| Rosehip oil | 3% w/w |
| Phenoxyethanol | 0.5% w/w |

The compositions are used at home or administered by a professional for facial rejuvenation. The composition is applied over the surface of the neck and face with circular movements until it has been completely absorbed, once daily at night. This composition has a repairing effect on the skin; it promotes collagen formation and elastic fibers, very useful to avoid the formation of abnormal scars and, in return, prevents the loss of water, thus increasing the hydration capacity of the skin surface, making it smooth and luminous. Use of this composition restructures the skin, protecting the skin from damage caused by free radicals. Use of the composition promotes hydration, activates the renewal of skin cells, helps to rejuvenate, supports photoprotection, promotes DNA reparation, and helps regenerate collagen and hydrate the skin.

### Example 14 - Eye Contour Gel

Skin care compositions are provided for rejuvenating mature skin or prematurely aging skin around the eyes, softening the lines around the eyes and reducing signs of fatigue. In some embodiments, the compositions include Double Distilled Water, Aloe vera Extract, Chamomile, Cell Growth Factors, Exthoxylated lanolin, Carbomer, Triethanolamine, Collagen, Elastin, Portulaca oleracea DNA, Phenoxythanol, and Fragrance/Aroma. In certain embodiments, the compositions include some of these components at the concentrations indicated in Table 12, with the other components making up the remainder of the composition. The compositions are for external use only.

TABLE 12

| | |
|---|---|
| Component | % w/w |
| Cell Growth Factors | 20% w/w |
| Chamomile | 10% w/w |
| Aloe Vera | 10% w/w |
| Phenoxyethanol | 0.5% w/w |

The compositions are used at home or administered by a professional for facial rejuvenation. The composition is applied over the surface of eye contour with circular movements until it has been completely absorbed, once daily at night. This composition is ultralight, fresh, and non-greasy. Use of the composition visibly reduces dark circles and puffiness to illuminate and relax the gaze. The composition is enriched with decongestant plant extracts in combination with growth factors, and its formulation helps to fight the signs of fatigue. The gaze is toned and regains its luminosity. This anti-dark circle treatment is suitable even for the most sensitive eyes.

### Example 15 - Sunscreen

Skin care compositions are provided for the daily solar protection of all skin types. In some embodiments, the compositions include Double Distilled Water, Oat Extract, Fat water soluble emollients, 2-Ethyl Hexyl Salicylate, 2-Ethyl Hexyl 4-Methoxycinnamate, 2-hydroxy-4-Methoxybenzophenone, Cell Growth Factors, Micronized Titanium Dioxide, Phenoxythanol, and Fragrance/Aroma. In certain embodiments, the compositions include some of these components at the concentrations indicated in Table 13, with the other components making up the remainder of the composition. The compositions are for external use only.

TABLE 13

| | |
|---|---|
| Component | % w/w |
| Oat | 40% w/w |
| Cell Growth Factors | 5% w/w |
| Phenoxyethanol | 0.5% w/w |

The compositions are applied over the surface of the neck and face with circular movements. The compositions are applied in the mornings prior to sun exposure or after excessive sweating. The compositions have the advantages of being imperceptible on the skin, having a non-greasy texture, being non-sticky, being quickly absorbed by the skin, providing maximum UVB /UVA protection, and being photostable. Use of the compositions helps to hydrate dry skin and strengthens the skin’s own protection system against free radicals, which are the main triggers of allergies to the sun.

### Example 16 - Revitalizing Skin Gel

Skin care compositions are provided for the treatment of sensitive or devitalized skin. In some embodiments, the compositions include Double Distilled Water, Cell Growth Factors, Mimosa Tenuiflora Extract, Pantenol, Manteca Karite, Glycerin, Propylene glycol, Xanthan Gum, Rosa Rubiginosa, Hyaluronic Acid, Aloe Extract, and Phenoxyethanol. In certain embodiments, the compositions include these components at the concentrations indicated in Table 14. The compositions are for external use only.

TABLE 14

| | |
|---|---|
| Component | % w/w |
| Distilled Water | 60.5% w/w |
| Conditioned Medium | 20% w/w |
| Mimosa Tenuiflora Extract | 5% w/w |
| Panthenol | 3% w/w |
| Shea Butter | 2% w/w |
| Glycerin | 1.5% w/w |
| Propylene glycol | 1.5% w/w |
| Xanthan Gum | 1.5% w/w |
| Rosa Rubiginosa essential oil | 1% w/w |
| Hyaluronic Acid | 1% w/w |
| Aloe Extract | 2% w/w |
| Phenoxyethanol | 1% w/w |

The compositions are used at home or administered by a professional for the treatment of sensitive skin and/or for a lifting effect. A portion of the composition is applied to clean and toned skin of the neck and face with circular movements until it has been completely absorbed. The composition is applied twice daily, in the morning and night. Use of the composition improves cell metabolism and softens and regenerates the elastic and collagen fibers of the skin tissue.

### Example 17 - Treatment of Skin With Topical Composition

Compositions comprising growth factors as disclosed herein were used in a twelve week study to evaluate the tolerability and efficacy on younger skin with acne and on older skin with photoaging, hyperpigmentation, and/or sunburn. Females of Fitzpatrick skin types I-VI who met all of the inclusion criteria (18-37 years of age with acne or 37-65 with photoaging, hyperpigmentation, and/or sunburn; all Fitzpatrick skin types, no known medical conditions that may interfere with study participation; willingness to cooperate with study requirements; signed informed consent; willing to use birth control during study) and none of the exclusion criteria were enrolled in the study. Exclusion criteria include subjects with one or more of the following: dermatological disorder that may interfere with accurate evaluation of subject’s skin; not willing to use the assigned study products as instructed; subjects who have had any facial treatments in the past 6 months and/or that are not willing to withhold facial treatments during the course of the study (facials, facial peels, laser treatments, dermabrasion, botulinum toxin (Botox), injectable filler treatments, intense pulsed light, acid treatments, tightening treatments, facial plastic surgery); uncontrolled systemic disease; significant history or current evidence of a medical, psychological, or other disorder; known hypersensitivity to any of the components of the study product; using a topical product containing a retinoid, retinol, or other vitamin A derivative within 3 months prior to or during the study period; using systemic steroid therapy; using any topical medicated creams, lotions, powders, etc. on the treatment areas during the study period, other than the study treatment regimen; using any topical sunless tanning products containing dihydroxyacetone (DHA) on the treatment areas for at least 7 days prior to the start of the study as well as throughout the entire course of the study; undergoes facial waxing, bleaching, or depilatory cream use within 30 days prior to entering the study as well as throughout the entire course of the study; has used any topical products containing alpha-hydroxy acids, salicylic acid, or vitamin C on the face for at least 7 days prior to the start of the study, as well as throughout the entire course of the study; pregnant, breast feeding, or planning a pregnancy; unwilling or unable to comply with the requirements of the protocol; history of a psychological illness or condition that would interfere with their ability to understand and follow the requirements of the study; currently participating in any other clinical trial; and/or having started hormone replacement therapies (HRT) or hormones for birth control less than 3 months prior to the study entry or who plan on starting, stopping or changing doses of HRT or hormones for birth control during the study.

Subjects were asked to continue their self-selected colored cosmetics unchanged throughout the 12-week study. Subjects used the study skin care products, and no other facial skin care products were used. Subjects were assigned to one of two groups. The characteristics of the 2 groups are listed below:
Group 1: Healthy female subjects 18-37 years of age with acne (15 subjects)
Group 2: Healthy female subjects 37-65 years of age with mild to moderate photoaging (15 subjects)

Subjects were provided with a daily compliance diary and study products appropriate for their group assignment, which was based on their skin care needs. The following products as disclosed in this application were used by the Group 1 study group (young biotype skin, mixed to oily, ages 18-37). In their daily morning regimen, the Group 1 subjects used Facial Dermo Cleanser, Hydrating Facial Toner, and Sunscreen. For their daily evening regimen, the Group 1 subjects used Facial Dermo Cleanser, Revitalizing Facial Serum, Eye Contour Gel, and Facial Moisturizing Gel. The following products as disclosed in this application were used by the Group 2 study group (mature biotype skin, dry skin, ages 37-65). In their daily morning regimen, the Group 2 subjects used Facial Dermo Cleanser, Hydrating Facial Toner, Sunscreen, and Hydrating Cream. For their daily evening regimen, the Group 2 subjects used Facial Dermo Cleanser, Nutritive Serum, Eye Contour Lifting Effect, Eye Contour Gel, and Nourishing Night Cream.

Subjects returned to the research center at week 2, week 4, week 8, and week 12 for the assessments described below. In addition, 3 subjects from each study group participated in a photography substudy. VISIA-CR images were taken of the front, right, and left face with standard lighting at baseline and week 12 (data not shown).

The dermatologist investigator and subjects assessed the following facial efficacy parameters: fine lines, wrinkles, texture, radiance, luminosity, smoothness, softness, skin tone evenness, firmness, pores, hyperpigmentation, and overall facial appearance. The acne group also assessed acne severity. All assessments were made on a 5-point ordinal scale (0=none, 1=minimal, 2=mild, 3=moderate, 4=severe). The assessments were made at baseline, week 2, week 4, week 8, and week 12.

The dermatologist investigator assessed tolerability in terms of peeling, dryness, redness, and swelling on the same 5-point ordinal scale (0=none, 1=minimal, 2=mild, 3=moderate, 4=severe) at baseline, week 2, week 4, week 8, and week 12. Finally, the subjects assessed tolerability in terms of itching, stinging, burning, and irritation on the same 5-point ordinal scale (0=none, 1=minimal, 2=mild, 3=moderate, 4=severe) at baseline, week 2, week 4, week 8, and week 12.

No adverse events or experiences occurred during the study, and only one subject withdrew from the study for personal reasons unrelated to product use. Along with descriptive statistics (means, standard deviations and percentages), investigator and subject ordinal nonparametric data were analyzed using the Wilcoxon signed rank test. Changes were considered significant at a p value of less than or equal to 0.05.

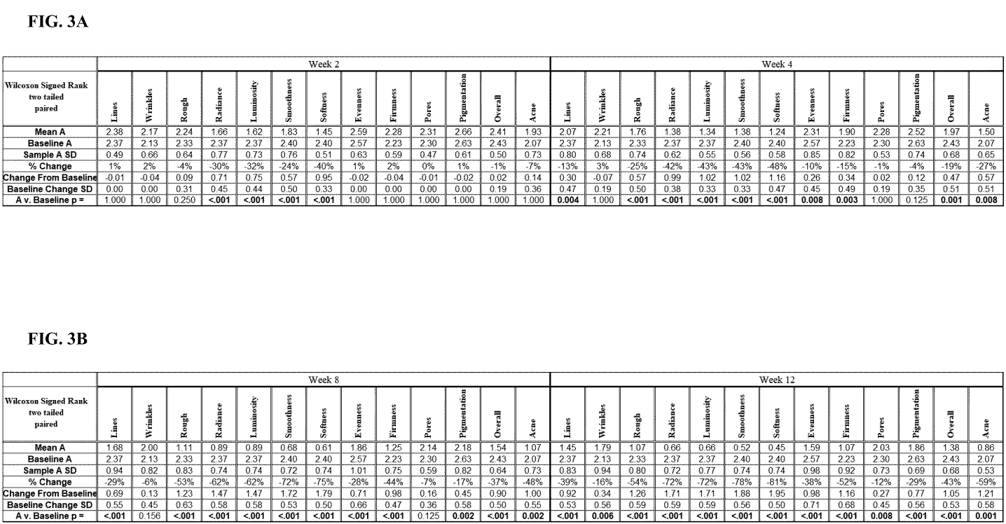

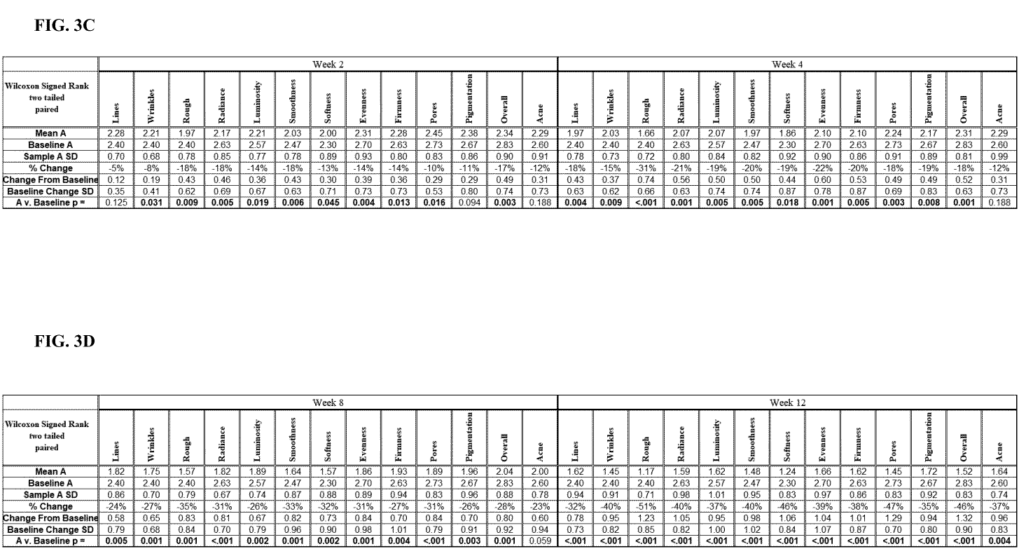

Exemplary data are shown in FIGS. 3A-3D. The investigator assessed statistically significant (p<0.001) improvement in radiance, luminosity, softness, and smoothness after 2 weeks of product use (FIG. 3A). Improvement continued into week 4 with statistically significant improvement lines, roughness, radiance, luminosity, smoothness, softness, evenness, firmness, and overall appearance (FIG. 3A). In addition, there was statistically significant (p=0.008) improvement in acne in the younger acne cohort. This cumulative improvement continued into week 8 with the following parameters showing statistically significant results: lines, roughness, radiance, luminosity, smoothness, softness, evenness, firmness, pigmentation, and overall appearance (FIG. 3B). The acne improvement continued to be statistically significant. Similar results were seen at week 12, with all parameters indicating statistically significant improvement: lines, wrinkles, roughness, radiance, luminosity, smoothness, softness, evenness, firmness, pigmentation, acne, and overall appearance (FIG. 3B). The investigator noted excellent anti-aging appearance benefits with the skin care regimen. The investigator noted no statistically significant tolerability issues and concluded the tolerability profile was excellent.

Similarly, the subjects rated statistically significant improvement from week 2 onward for all evaluated criteria except lines, pigmentation, and acne (FIG. 3C). All criteria, except for acne, were significantly improved at weeks 4 and 8 (FIGS. 3C-3D). By week 12, all criteria were highly statistically significant (p<0.001) as rated by the subjects, including acne (FIG. 3D). No statistically significant tolerability issues were identified by the subjects at any point during the study. The subject tolerability profile was excellent.

### Example 18 - Treatment of Wound With Topical Composition

Patients presented with an open wound that was in need of treatment. The patients were treated with an effective amount of a composition that includes exosomes, growth factors, DMDM hidantoin, phenoxyethanol, benzyl alcohol, and fragments of mesenchymal stem cells (MSCs). The composition was in a gel, cream, lotion, serum, or spray formulation and was administered directly to the skin surrounding the wound. The composition promoted healing of the wound and shortened the healing period (data not shown).

### Example 19 - Treatment of Wound With Injectable Composition

Patients presented with an open wound that was in need of treatment. The patients were treated with an effective amount of a composition that includes exosomes, growth factors, DMDM hidantoin, phenoxyethanol, benzyl alcohol, and fragments of mesenchymal stem cells (MSCs). The composition was in solution and was administered by intradermal injection to the skin at or surrounding the wound. The composition promoted healing of the wound and shortened the healing period.

The above examples are provided to illustrate the disclosure but not to limit its scope. Other variants of the disclosure will be readily apparent to one of ordinary skill in the art and are encompassed by the appended claims. All publications, databases, internet sources, patents, patent applications, and accession numbers cited herein are hereby incorporated by reference in their entireties for all purposes.

What is claimed is:

1. A composition for cosmetic or therapeutic use, comprising:
a. exosomes,
b. growth factors,
c. optionally DMDM hidantoin,
d. optionally phenoxyethanol,
e. optionally benzyl alcohol, and
f. fragments of mesenchymal stem cells (MSCs).

2. The composition of claim 1, wherein the composition comprises exosomes at a concentration of about 55 exosomes/µL.

3. The composition of claim 1, wherein the composition comprises DMDM hidantoin at 0.2% w/w of the composition, phenoxyethanol at 0.3% w/w of the composition, and benzyl alcohol at 0.3% w/w of the composition.

4. The composition of claim 1, wherein the growth factors comprise VEGF, FGF, IL-6, CTGF, PDGF, PGE2, IL-10, HLA-G5, galectin 1, LIF, MHCII, EGF, TGF-β, IGF-1, KGF, SDF-1, MIP-1a, MIP-1b, SDF-1, or HGF, or a combination thereof.

5. The composition of claim 1, wherein the composition comprises double distilled water, growth factors, placenta hydrolyzate, Portulaca oloracea DNA, a water soluble emollient, cellulose, phenoxyethanol, and a fragrance.

6. The composition of claim 1, wherein the composition comprises double distilled water, growth factor, portulaca oleracea extract, a water soluble emollient, an exopolysaccharide, collagen, sodium L-Pyrrolidonecarboxylate (Sodium PCA), cellulose, elastin, thriethanolamine, phenoxyethanol, and a fragrance.

7. The composition of claim 1, wherein the composition comprises double distilled water, growth factor, portulaca oleracea extract, vegetable glycerin, polysorbate 20, Portulaca oloracea DNA, phenoxyethanol, and a fragrance.

8. The composition of claim 1, wherein the composition comprises double distilled water, growth factor, portulaca oleracea extract, a water soluble emollient, Portulaca oloracea DNA, collagen, sodium L-Pyrrolidonecarboxylate (sodium PCA), cellulose, elastin, phenoxyethanol, and a fragrance.

9. The composition of claim 1, wherein the composition comprises double distilled water, growth factor, lauryl glucoside, disodium cocoamphodiacetate, ethoxylated lanonin, cellulose, phenoxyethanol, and a fragrance.

10. The composition of claim 1, wherein the composition comprises ionized mineral water, growth factor, a non-greasy emollient, polysorbate 20, vetiver essential oil, and phenoxyethanol.

11. The composition of claim 1, wherein the composition comprises double distilled water, growth factor, hyaluronic acid, anhydrous lanolin, lauryl glucoside, a non-greasy emollient, hydroxmethylcellulose, phenoxyethanol, and a fragrance.

12. The composition of claim 1, wherein the composition comprises double distilled water, growth factor, synergy organic oils of Olea europaea, argania spinosa, vitamin E, Portulaca oloracea DNA, phenoxyethanol, and a fragrance.

13. The composition of claim 1, wherein the composition comprises oat extract, growth factor, fatty emollients emulsified with water, collagen, elastin, vitamin E, phenoxyethanol, and a fragrance.

14. The composition of claim 1, wherein the composition comprises double distilled water, growth factor, mixture of fatty emollients emulsified with water, organic roseship oil, vitamin E, phenoxyethanol, and a fragrance.

15. The composition of claim 1, wherein the composition comprises double distilled water, aloe vera extract, chamomile, growth factor, ethoxylated lanolin, carbomer, triehtanolamine, collagen, elastin, Portulaca oloracea DNA, phenoxyethanol, and a fragrance.

16. The composition of claim 1, wherein the composition comprises double distilled water, oat extract, fat water soluble emollients, 2-ethyl hexyl salicylate, 2-ethyl hexyl 4-methoxycinnamate, 2-hydroxy-4-methoxybenzophenone, growth factor, micronized titanium dioxide, phenoxyethanol, and a fragrance.

17. The composition of claim 1, wherein the composition comprises distilled water, cell growth factors, mimosa tenuiflora extract, panthenol, shea butter, glycerin, propyleneglycol, xanthan gum, rosa rubiginosa essential oil, hyaluronic acid, aloe extract, and phenoxyethanol.

18. A method of producing a composition for cosmetic or therapeutic use, comprising:
a. culturing mesenchymal cells (MSCs) in a suitable culture medium for 96 hours to 3 months;
b. centrifuging the culture of MSCs;
c. carefully removing the supernatant from the centrifuged culture of MSCs;
d. using flow cytometry to obtain a fraction of the supernatant that comprises growth factors, exosomes, and fragments of MSCs; and
e. adding one or more pharmaceutically acceptable excipients to the fraction, thereby producing the composition for cosmetic or therapeutic use.

19. The method of claim 18, wherein the one or more pharmaceutically acceptable excipients comprise DMDM hidantoin, phenoxyethanol, benzyl alcohol, or a combination thereof.

20. A composition produced by the method of claim 18.

21. The composition of claim 1, wherein the composition is formulated as a gel, cream, lotion, serum, spray, patch, or solution.

22. A method of treating an individual in need thereof by administering an effective amount of the composition of claim 1 to the individual.

23. The method of claim 22, wherein the composition is administered topically.

24. The method of claim 22, wherein the individual is in need of skin revitalization or solar protection.

25. The method of claim 22, wherein the effective amount promotes hydration of the skin, stimulates production of collagen, increases elasticity of the skin, regulates the amount of skin fat, neutralizes free radicals, acts as a decongestant, decreases inflammation, or a combination thereof.

26. The method of claim 22, wherein the individual has a wound, burn, or other skin injury.
